(19) United States
(12) Patent Application Publication
(10) Pub. No.: US 2026/0347743 A1
(43) Pub. Date: Nov. 5, 2026

(54) Specimen Analysis Device

(71) Applicant: Hitachi High-Tech Corporation, Minato-ku, Tokyo (JP)

(72) Inventors: Tadao YABUHARA, Tokyo (JP); Jin MATSUMURA, Tokyo (JP)

(21) Appl. No.: 19/109,186

(22) PCT Filed: Sep. 14, 2022
(86) PCT No.: PCT/JP2022/034311

§ 371 Date: Mar. 6, 2025

(51) Int. Cl. C12M 1/34; C12M 1/00

(52) U.S. Cl. CPC C12M 41/12; C12M 23/38

(57) ABSTRACT

The purpose of the present invention is to precisely control the temperature in each storage chamber of a specimen analysis device in which a plurality of housing chambers house specimen containers, respectively. The specimen analysis device according to the present invention generates cold air (see FIG. 2) such that the temperature of the specimen container lower portion is lower than the temperature of a specimen container lid.

### TECHNICAL FIELD

The present invention relates to a specimen analysis device that analyzes a specimen.

### BACKGROUND ART

In a medical research institute, a hospital, or the like, a culture state of cells or bacteria is inspected based on a turbidity of a specimen including cells or bacteria. In the cell culture or the bacteria culture, a microwell plate, a culture dish, or the like is used as a specimen container. A pre-treated specimen, nutrient, or the like is dispensed or applied into a specimen container, and the specimen is cultured (for example, in an environment of 35 °C). Regarding the specimen in the specimen container, culture and observation are repeated for a long period of time to quantify a change in the shape of the specimen during the observation through image analysis or based on a turbidity, and the result is output according to a quantitative value thereof or the amount of change over time.

In this inspection, the specimen container is provided in the device for a long period of time. The inside of the specimen container enters a saturated state due to evaporation of a culture solution or the like. Therefore, dew condensation is likely to occur on a boundary surface between the specimen container and a specimen container lid. In an inspection device (transmission observation) that optically observes the specimen in the specimen container from the outside of the specimen container, when dew condensation occurs on a transmission member surface such as a lid of a specimen container upper surface that is interposed, refraction of light occurs, which leads to deterioration in contrast and a decrease in light intensity. In addition, it is difficult to accurately observe a change in the state of the specimen, which leads to erroneous determination of the measurement result.

PTL 1 describes a temperature control when a specimen is observed using a microscope. PTL 1 discloses a technique of blowing warm air to an observation tray upper portion to prevent occurrence of dew condensation on an observation tray lid inner surface.

### CITATION LIST

### Patent Literature

PTL 1: JP4116780B

### SUMMARY OF INVENTION

### Technical Problem

A specimen analysis device places a specimen container accommodating a specimen (cells, bacteria, or the like) on each of a plurality of housing chambers, and regularly observes the specimen while culturing the specimen. When dew condensation occurs in a lid of the specimen container, the light intensity of observation light is likely to decrease due to the dew condensation such that the measurement accuracy may decrease. Accordingly, a mechanism for preventing dew condensation is required. For example, as in PTL 1, the supply of warm air to the specimen container is considered.

However, when the effect of the warm air on each of the housing chambers varies, the effect of suppressing dew condensation also varies depending on each of the housing chambers. Due to this variation, the accuracy of the temperature control may decrease depending on each of the housing chambers, and a variation in the measurement result is likely to occur.

The present invention has been made in consideration of the above-described problems, and an object thereof is to accurately control a temperature in each of a plurality of housing chambers in a specimen analysis device where each of the housing chambers houses a specimen container.

### Solution to Problem

With a specimen analysis device according to the present invention, cold air is generated such that a temperature of a specimen container lower portion is lower than a temperature of a specimen container lid.

### Advantageous Effects of Invention

In the specimen analysis device according to the present invention, it is possible to accurately control a temperature in each of a plurality of housing chambers when each of the housing chambers houses a specimen container. The other configurations, tasks, effects, and the like of the present invention will be clarified from the description of the following embodiments.

### BRIEF DESCRIPTION OF DRAWINGS

FIG. 1 is a schematic diagram illustrating an overall configuration of an analysis device 0001 according to a first embodiment.

FIG. 2 illustrates a configuration example of a housing section 0003.

FIG. 3 illustrates a shape example of an air volume adjusting material 2116 and an outlet 2113.

FIG. 4 illustrates another configuration example of the air volume adjusting material 2116.

FIG. 5 illustrates a configuration example of the housing section 0003 in the analysis device 0001 according to a second embodiment.

FIG. 6 illustrates another configuration example of the housing section 0003.

FIG. 7 illustrates another configuration example of the housing section 0003.

FIG. 8 illustrates another configuration example of the housing section 0003.

FIG. 9 illustrates another configuration example of the housing section 0003.

FIG. 10 is a flowchart illustrating a procedure where an arithmetic section 11 controls a temperature of each of sections in the analysis device 0001.

FIG. 11 is a flowchart illustrating a procedure where the arithmetic section 11 controls a temperature of each of sections in the analysis device 0001.

### DESCRIPTION OF EMBODIMENTS

### First Embodiment

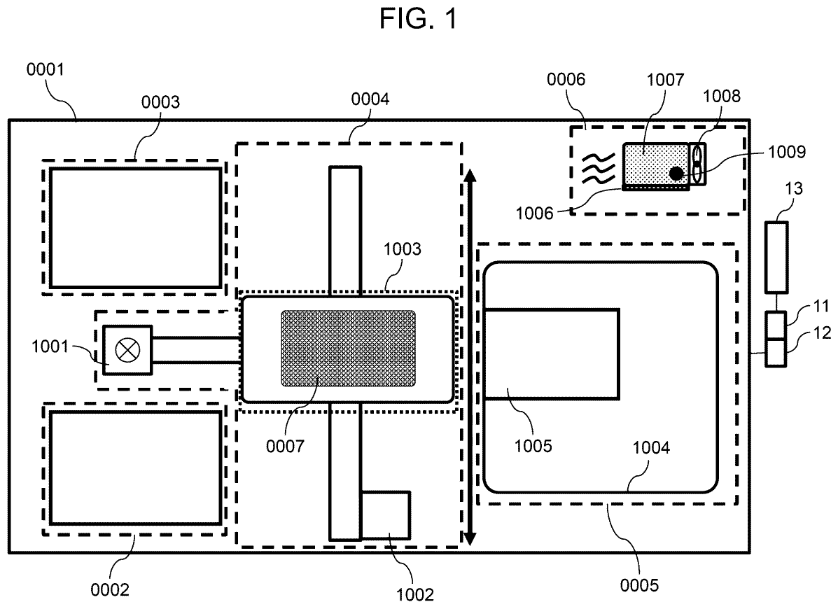

FIG. 1 is a schematic diagram illustrating an overall configuration of an analysis device 0001 according to a first embodiment of the present invention. The analysis device 0001 includes a loading/unloading section 0002, a housing section 0003, a transport section 0004, a detection section 0005, and a temperature control section 0006. An arithmetic section 11, a storage section 12, and a monitor 13 can be provided outside the analysis device 0001 or can be provided inside the analysis device 0001.

In the loading/unloading section 0002, a user can load and unload a specimen container 0007 through a door (not shown). The specimen container 0007 includes two components including a specimen container lower portion 2110 and a specimen container lid 2112 described below. The specimen container lower portion 2110 is a container including a plurality of wells such as 96 wells or 384 wells, and a specimen 2111 is taken in each of the wells. Examples of the specimen include a biological body such as a cell, blood, urine, a bacterium, or a piece of tissue. The specimen container lid 2112 may have a seal shape, or the specimen container lower portion 2110 may be a single well.

The housing section 0003 includes plural stages of specimen container housing chambers 2003 each of which houses the specimen container 0007. The analysis device 0001 may include a plurality of housing sections 0003. The details of the housing section 0003 will be described below.

The transport section 0004 includes an actuator 1001, an actuator 1002, a specimen container holding section 1003, and a ball screw or a belt mechanism (not illustrated). The specimen container holding section 1003 is moved by the actuator 1001 in the vertical direction through the ball screw or the belt mechanism, and is moved in a depth direction by the actuator 1002. The specimen container holding section 1003 can receive and deliver the specimen container 0007 from the loading/unloading section 0002, the housing section 0003, and a measurement section 1005.

In the detection section 0005, the measurement section 1005 in a measurement unit 1004 receives the specimen container 0007 from the specimen container holding section 1003, and measures a culture state of the specimen 2111 in each of the wells of the specimen container 0007. Examples of the measurement method include turbidity measurement, absorbance measurement, fluorescence measurement, and image analysis.

The temperature control section 0006 includes a heat source 1006, a heat sink 1007, and a fan 1008. Heat of the heat source 1006 through the heat sink 1007 is supplied into the device through air from the fan 1008. As the heat source, a heater, a Peltier element, or the like is used to increase or decrease the temperature in the analysis device 0001. As the heat sink (heat dissipator), aluminum, copper, iron, stainless steel, or the like can be used. The temperature control section 0006 has a function of warming the entire analysis device 0001 such that dew condensation described below does not occur when the specimen container 0007 is taken out from the housing section 0003.

When the analysis device 0001 starts, the user can provide the specimen container 0007 in the loading/unloading section 0002. After being provided, the specimen container 0007 of the loading/unloading section 0002 is transported to the measurement section 1005 of the measurement unit 1004 through the transport section 0004. The measurement section 1005 measures the culture state of the specimen 2111 in the specimen container 0007. After the measurement, the specimen container 0007 is transported to the housing section 0003 through the transport section 0004. A measurement cycle of the specimen is repeated for about 18 hours, for example, at intervals of 20 to 30 minutes at a maximum. The analysis device 0001 transmits the amount of change over time in the culture state of the specimen 2111 to the arithmetic section 11. The arithmetic section 11 outputs the measurement result estimated from the amount of change to the monitor 13 or the like. After completion of the measurement, the specimen container 0007 is unloaded to the loading/unloading section 0002 through the transport section 0004. In the present embodiment, the user provides the specimen container 0007 in the loading/unloading section 0002. However, a structure where the user directly provides the specimen container 0007 in the housing section 0003 may be adopted instead of providing the loading/unloading section 0002.

As described above, the specimen container 0007 includes the specimen container lower portion 2110 and the specimen container lid 2112. The specimen 2111 is taken in each of the wells of the specimen container lower portion 2110. In a case where the specimen container 0007 is provided in the housing section 0003, when thermal energy supplied to the specimen 2111 in the specimen container lower portion 2110 from a material, air, and the like of a lower surface of the specimen container lower portion 2110 is higher than thermal energy supplied to the specimen container lid 2112, dew condensation occurs on a boundary surface between the specimen container lower portion 2110 and the specimen container lid 2112.

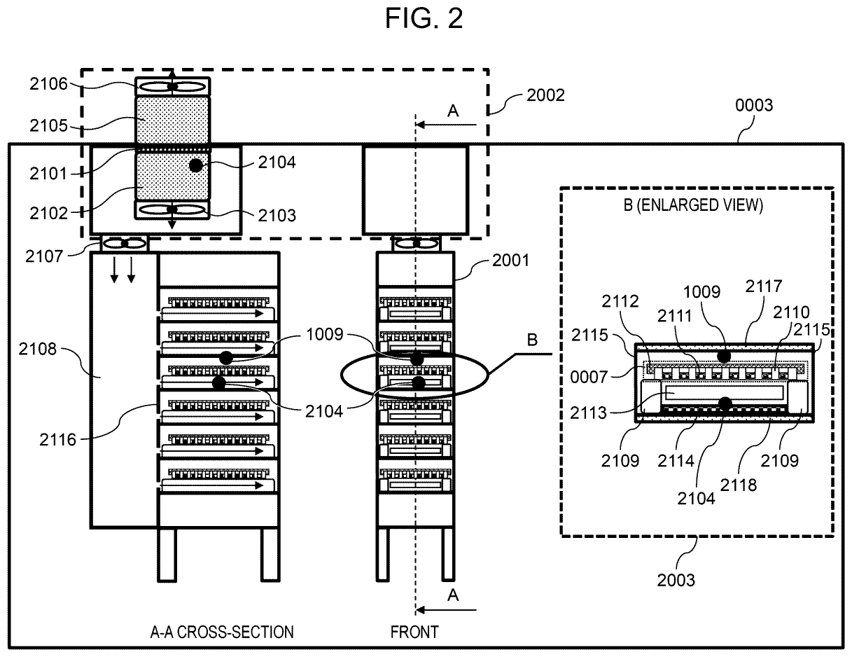

FIG. 2 illustrates a configuration example of the housing section 0003. The housing section 0003 includes a housing unit 2001 and a temperature control section 2002. The housing unit 2001 includes the specimen container housing chamber 2003, a fan 2107, a duct 2108, and an air volume adjusting material 2116. The housing section 0003 includes plural stages of specimen container housing chambers 2003, and each of the specimen container housing chambers 2003 houses the specimen container 0007. In the drawing, the number of stages of the specimen container housing chambers 2003 is six. However, the number of the stages is not limited to this example and may be increased or decreased, and the housing chambers may be arranged in the horizontal direction.

The specimen container housing chamber 2003 is surrounded by a heat insulating material 2109, the air volume adjusting material 2116, a heat insulating material 2114, a side-surface metal material 2115, an upper-surface metal material 2117, and a lower-surface metal material 2118. The air volume adjusting material 2116 includes an outlet 2113. Examples of the metal material include aluminum, stainless steel, copper, iron, and titanium. Examples of the heat insulating material include glass wool, cellulose fiber, an insulation board, a wool heat insulating material, rock wool, rigid urethane foam, bead polystyrene foam, and phenol foam, but the heat insulating material is not limited thereto. A resin may be used as the heat insulating material. Examples of the resin include nylon, POM, PEEK, PPS, PTFE, PVC, PE, PP, PS, and ABS, but the resin is not limited thereto.

The temperature control section 2002 includes a cold source 2101, a heat sink 2102, a temperature sensor 2104, a fan 2103, a heat sink 2105, and a fan 2106. Thermal energy of the cold source 2101 through the heat sink 2102 is supplied into the housing unit 2001 through air from the fan 2103. As the cold source 2101, a heater, a chiller, a Peltier element, or the like can be used. The temperature sensor 2104 attached to the heat sink 2102 controls heating or cooling temperature. As the heat sink (heat dissipator or heat dissipation plate), aluminum, copper, iron, stainless steel, or the like can be used. A thermistor, a platinum resistor, an IC chip, a thermocouple, or the like may be used as the temperature sensor 2104, and an installation location thereof may be an installation space of the temperature control section, the inside of the duct 2108, or the like in addition to the heat sink 2102.

The heat sink 2105 and the fan 2106 are configurations assuming a Peltier element, and configure a heat dissipation side of the Peltier element. The heat sink 2105 of the heat dissipation side is provided in a housing of the analysis device 0001, which may be used as an auxiliary heat source for controlling the temperature in the device. When the temperature control is difficult, a structure where the heat sink 2105 is provided in an outer space (an examination room or a laboratory) of the analysis device 0001 to discharge heat to the outside of the device may be adopted.

Cold air controlled by the temperature control section 2002 is supplied into the duct 2108 through the fan 2107. The cold air supplied into the duct 2108 is supplied onto the lower surface of the specimen container lower portion 2110 through the outlet 2113 of the specimen container housing chamber 2003. By controlling the temperature such that the ambient temperature of the transport section 0004>the temperature of the cold air from the temperature control section 2002 is satisfied, the temperature of the specimen container lid 2112>the temperature of the specimen 2111 in the specimen container lower portion 2110 is satisfied, and dew condensation on the boundary surface between the specimen container lower portion 2110 and the specimen container lid 2112 is prevented.

For example, when the specimen 2111 is bacteria, there are bacterial species where growth is affected at 36 °C or higher and culture is delayed at 34 °C or lower. With the present configuration, the temperatures of the temperature control section 0006 and the temperature control section 2002 can be controlled such that the temperature of the housing unit 2001 is a temperature (for example, 35±1 °C) that does not affect the culture of the specimen 2111.

It is desirable that the position of the outlet 2113 is disposed between a bottom surface of the specimen container lower portion 2110 and an upper surface of the heat insulating material 2114 in the vertical direction and is disposed between the left and right heat insulating materials 2109 in the left-right direction such that the temperature of the bottom surface of the specimen container lower portion 2110 can reach a target temperature.

It is desirable that the cold air supplied from the duct 2108 in each of the stages is not supplied from above the specimen container lower portion 2110 of each of the stages. Alternatively, a relationship “an air volume of a lower portion of the specimen container 0007 >an air volume of an upper portion thereof≥0” may be satisfied to satisfy “the temperature of the specimen container lid 2112>the temperature of the specimen container lower portion 2110”. The warm air may flow from the lower portion to the upper portion of the specimen container 0007.

It is preferable that a position of a temperature sensor 1009 that controls the temperature control section 0006 is a position where a temperature of the specimen container lid 2112 or a space in the vicinity thereof can be relatively measured. In addition, as long as the correlation with the temperature of the specimen container lid 2112 in the housing section can be obtained, the temperature sensor 1009 may be positioned at any position. For example, the temperature sensor 1009 may be positioned in the heat sink 1007 and the vicinity thereof or in the vicinity of the specimen container lid 2112. The position of the temperature sensor 1009 or the number thereof is not limited.

It is preferable that a position of the temperature sensor 2104 that controls the temperature control section 2002 is a position where a temperature of the specimen container lower portion 2110 or a space in the vicinity thereof can be relatively measured. In addition, as long as the correlation with the temperature of the specimen container lower portion 2110 in the housing section can be obtained, the temperature sensor 2104 may be positioned at any position. For example, the temperature sensor 2104 may be positioned in the heat sink 2102 and the vicinity thereof or in the vicinity of the specimen container lower portion 2110. The temperature sensor 2104 may be positioned in the temperature control section 2002, in the duct 2108, in a space in the vicinity of an air volume adjusting material 2119 (described below), or the like. The position of the temperature sensor 2104 or the number thereof is not limited.

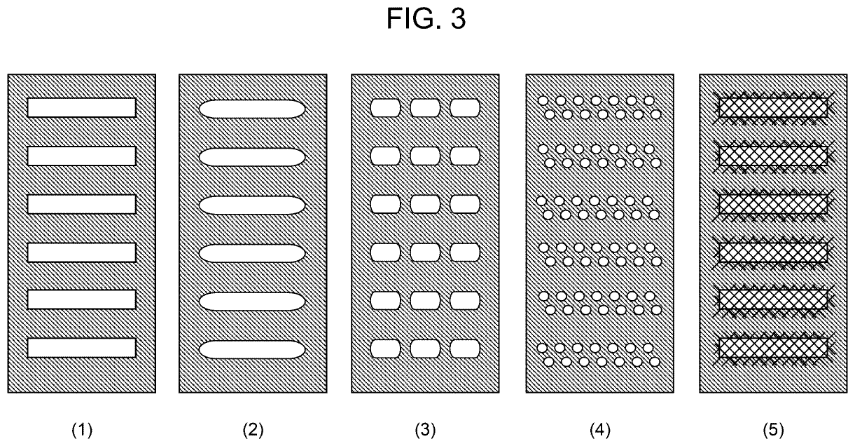

FIG. 3 illustrates a shape example of the air volume adjusting material 2116 and the outlet 2113. In order to uniformly supply the cold air from the upper stage to the lower stage of the specimen container housing chamber 2003, the air volume adjusting material 2116 includes the outlet 2113. The outlet 2113 may be an angle hole, a hole of an ellipse, a circle, or the like, a porous hole, or the like. That is, the shape of the outlet 2113 is not limited as long as the air volume flowing to the housing chamber can be controlled. The air volume blown from the outlet 2113 of the lower stage and the air volume blown from the outlet 2113 of the upper stage are made uniform by the air volume adjusting material 2116 such that the amount of evaporation of the specimen 2111 in the specimen container 0007 provided in the specimen container housing chamber 2003 is uniform across the upper stage to the lower stage (a variation in the amount of evaporation is suppressed). By suppressing the variation in the amount of evaporation, a change in the concentration of a culture solution, a chemical, or the like and a variation in the culture state of the specimen can be suppressed, and the erroneous determination risk of the inspection result can be reduced.

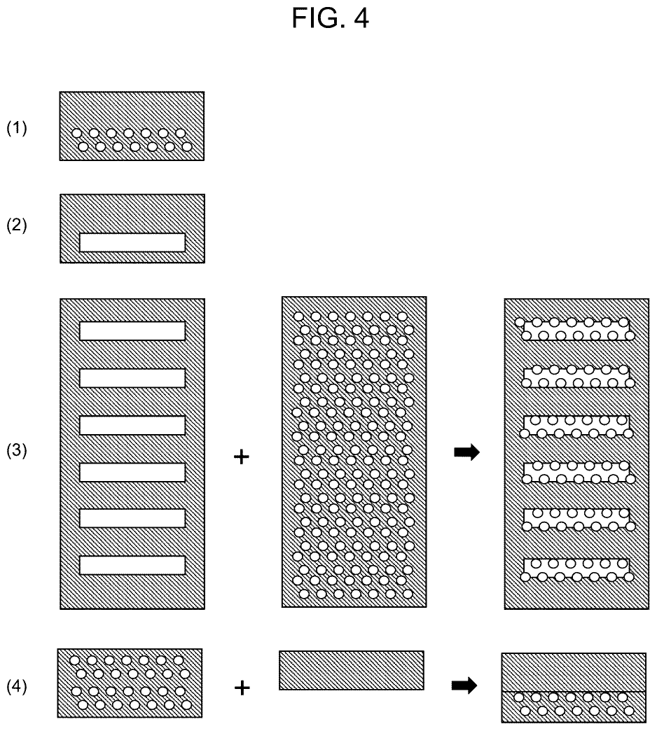

FIG. 4 illustrates another configuration example of the air volume adjusting material 2116. The air volume adjusting material 2116 may include a circular punched metal or a mesh-shaped portion. As long as an opening ratio can be controlled, the air volume adjusting material 2116 may be a continuous porous body or may be a material cut in a honeycomb structure, a quadrangular shape, or the like. Regarding the air volumes supplied to the upper portion and the lower portion of the plate in each of the stages, it is desirable that the upper portion in each of the stages has no or a small number of holes such that the air volume of the lower portion>the air volume of the upper portion≥0 is satisfied within a range where the temperature of the specimen container lid 2112>the temperature of the specimen container lower portion 2110 described above is satisfied. The air volume adjusting materials 2116 in the respective stages may have the same shape or different shapes depending each of the stages. In addition, in order to adjust the air volume, a member having one hole and a member having another hole may be combined. That is, the size/the number/a combination of arrangement of openings may be adjusted such that the air volume of a region below the specimen container lid 2112 is more than the air volume of a region above the specimen container lid 2112.

The heat insulating material 2114 is to reduce loss of thermal energy in the cold air supplied to the entire bottom surface of the specimen container lower portion 2110 and to prevent a decrease in the temperature of the metal material 2118 in the vicinity of the specimen container lid 2112 of the lower stage. Note that, when a temperature difference is sufficient between the upper and lower surfaces of the specimen container 0007, the heat insulating material 2114 does not need to be provided.

### First Embodiment: Summary

The analysis device 0001 according to the present embodiment includes the air volume adjusting material 2116 such that the cold air for making the temperature of the specimen container lid 2112 higher than the temperature of the specimen container lower portion 2110 is uniformly supplied to each of the specimen container housing chambers 2003. As a result, while using the simple configuration, the effect of suppressing dew condensation in each of the housing chambers can be made uniform. Accordingly, a variation in the measurement result in each of the housing chambers can be suppressed. In addition, the plurality of specimen containers 0007 can be accommodated and cultured, and improvement in the processing capacity of the device and a reduction in the burden of the user can be implemented. Further, by disposing the specimen containers 0007 together in the housing section 0003, the device size can be reduced.

In the analysis device 0001 according to the present embodiment, by combining the warm air supplied from the temperature control section 0006 and the cold air supplied from the temperature control section 2002, the temperature of the specimen 2111 can be accurately controlled. That is, both of a temperature increase by the warm air and a temperature decrease by the cold air can also be achieved. Therefore, the temperature can be controlled more accurately as compared to a configuration where only warm air is supplied.

### Second Embodiment

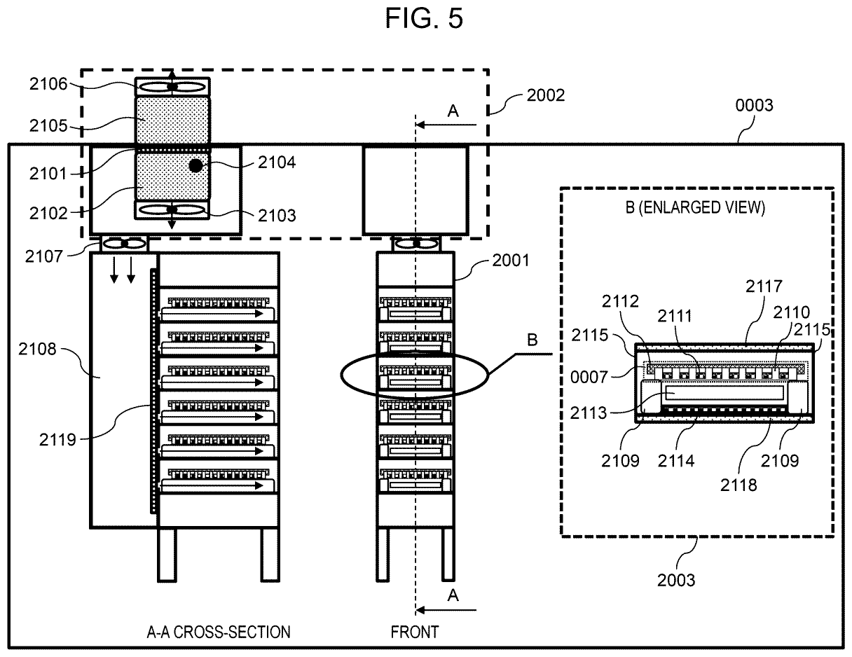

FIG. 5 illustrates a configuration example of the housing section 0003 in the analysis device 0001 according to a second embodiment of the present invention. In the configuration example shown in FIG. 5, in order to uniformly supply the cold air across the upper stage to the lower stage in the housing section 0003, the air volume adjusting material 2119 is provided in the vicinity of the outlet 2113. Other configurations are the same as those of the first embodiment.

When the air volume adjusting material 2119 is not used, the air volume of the outlet 2113 of the lower stage>the air volume of the outlet 2113 of the upper stage is likely to be satisfied. As a result, the amount of evaporation of the specimen 2111 in the specimen container 0007 provided in the specimen container housing chamber 2003 varies between the upper stage and the lower stage. That is, the concentration of a culture solution, a chemical, or the like varies, and the culture state of the specimen also varies. Therefore, there is a risk of leading to erroneous determination of the inspection result. The air volume adjusting material 2119 can further uniformize the air volume of the cold air across the upper stage to the lower stage as compared to the first embodiment, and thus the risk can be reduced. In general, the air volume adjusting material 2119 may include a circular punched metal or a mesh-shaped portion. As long as an opening ratio can be controlled, the air volume adjusting material 2119 may be a continuous porous body or may be a material cut in shape such as a honeycomb structure or a quadrangular shape.

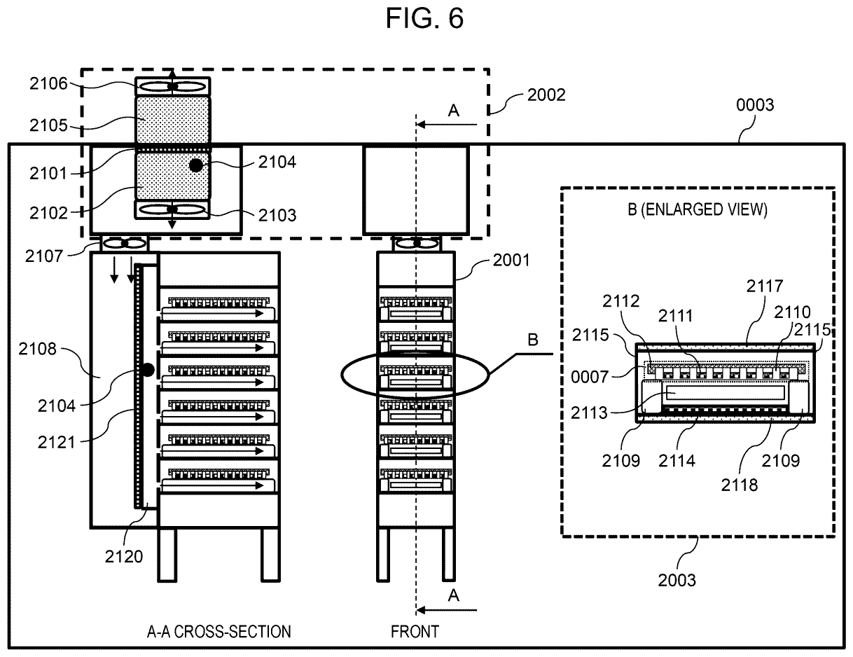

FIG. 6 illustrates another configuration example of the housing section 0003. In the configuration example illustrated in FIG. 6, a duct 2120 is provided in the duct 2108. An air volume adjusting material 2121 is disposed on a boundary surface between the duct 2108 and the duct 2120. In an inlet portion of the housing chamber in each of the stages, the air volume adjusting material 2116 is disposed as in the first embodiment. The air volume adjusting materials 2116 and 2121 may have the same configuration or may have different opening ratios (for example, the air volume adjusting material 2121 has a higher opening ratio). Other configurations are the same as those of the first embodiment.

The duct 2120 has an action of adjusting a pressure of the cold air. By providing the duct 2120, the influence of an axial flow of the fan 2107 can be suppressed, and a flow rate from the outlet 2113 can be uniformized across the upper stage to the lower stage. As a result, the amount of evaporation of the specimen 2111 in the specimen container 0007 can be fixed across the upper stage to the lower stage. The temperature sensor 2104 may be disposed on a surface or the like of the air volume adjusting material 2121 to control the temperature of the cold air.

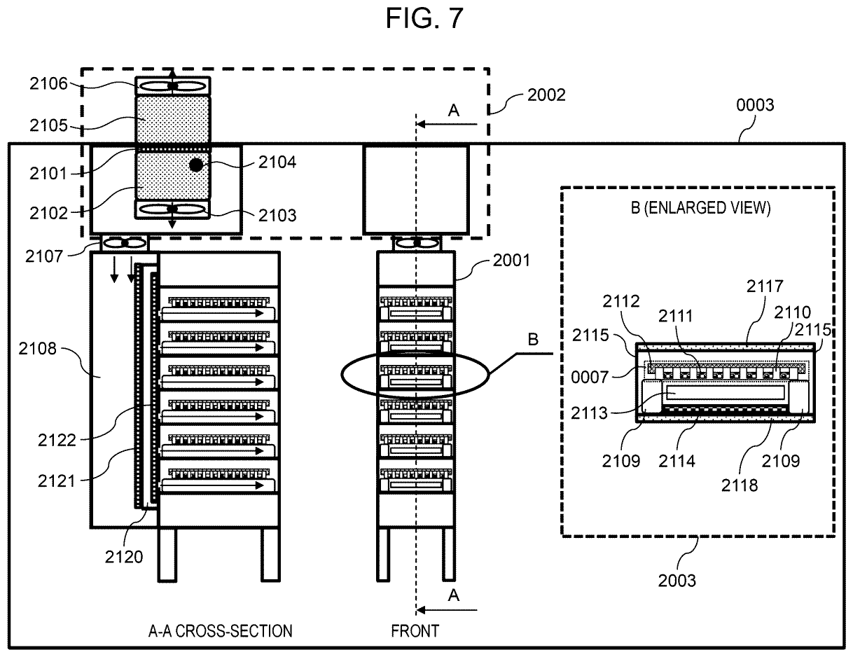

FIG. 7 illustrates another configuration example of the housing section 0003. In the configuration example illustrated in FIG. 7, in addition to the configuration described with reference to FIG. 6, an air volume adjusting material 2122 is provided on a boundary surface between the duct 2120 and the outlet 2113. Other configurations are the same as those of the first embodiment. By satisfying an opening ratio of the air volume adjusting material 2122<an opening ratio of the air volume adjusting material 2121, a flow rate of the air supplied from the outlet 2113 can be made more uniform across the upper stage to the lower stage as compared to FIG. 6.

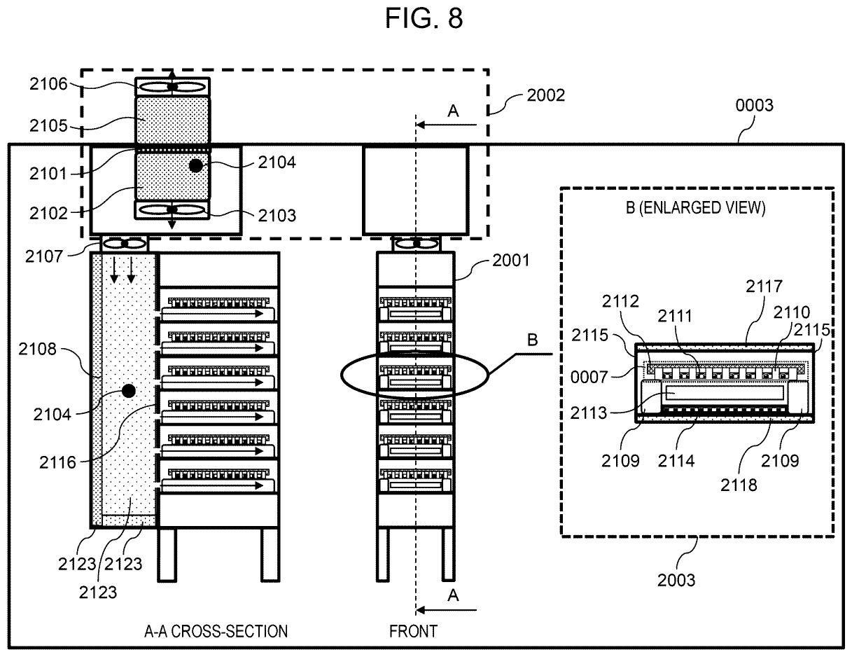

FIG. 8 illustrates another configuration example of the housing section 0003. In the configuration example illustrated in FIG. 8, a heat insulating material 2123 is attached to at least a part of an inner surface of the duct 2108 (and/or at least a part of an outer surface of the duct 2108). As a result, robustness to the temperature from the outside of the housing unit 2001 is improved. Other configurations are the same as those of the first embodiment. With the present structure, the cold air from the temperature control section 2002 where heat quantity loss is minimized can be supplied from the outlet 2113 to the bottom surface of the specimen container lower portion 2110. In the structure of any of FIGS. 5 and 6, the heat insulating material 2123 may be combined with this structure.

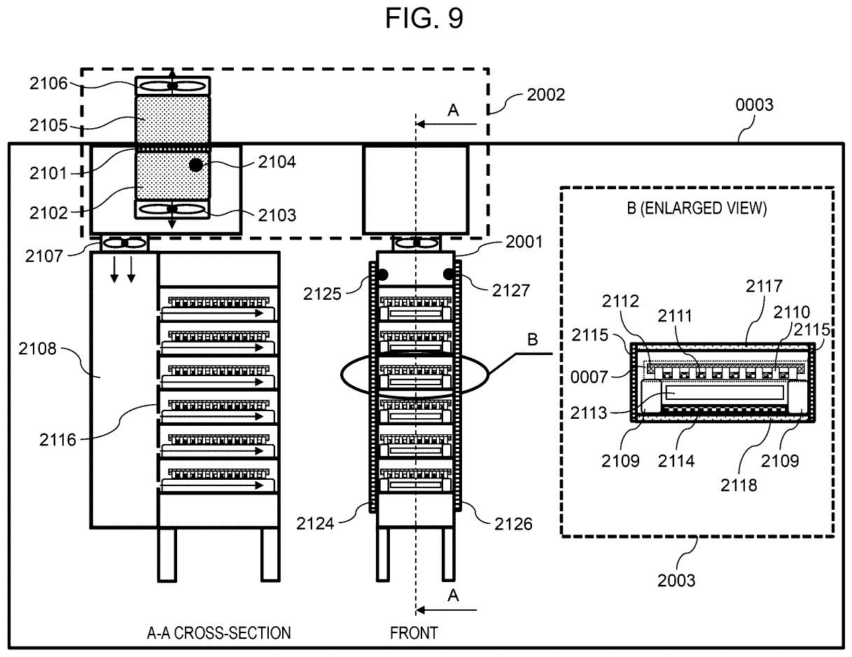

FIG. 9 illustrates another configuration example of the housing section 0003. In the configuration example illustrated in FIG. 9, a heat source 2124, a temperature sensor 2125 for controlling the temperature of the heat source 2124, a heat source 2126, and a temperature sensor 2127 for controlling the temperature of the heat source 2126 are provided on left and right side surfaces of the specimen container housing chamber 2003. As the heat sources 2124 and 2126, a heater, a Peltier element, or the like is used. Other configurations are the same as those of the first embodiment. This configuration may be combined with the configurations of FIGS. 5 to 8. The side surfaces described herein refer to at least surfaces that are not orthogonal to a path where the cold air passes through the inside of the specimen container housing chamber 2003.

For example, when the specimen 2111 is bacteria, the specimen container 0007 that is kept warm in the housing section 0003 needs to be kept warm at about 35 °C±1 °C for culturing the specimen 2111. The heat source 2124 and the heat source 2126 assist heating and are used for increasing the temperature control accuracy. In order to suppress dew condensation, controlled temperatures of the heat source 2124 and the heat source 2126>a controlled temperature of the cold source 2101 is satisfied, and the temperature of the cold air blown to the bottom surface of the specimen container lower portion 2110 is set to be lower than a warming temperature of the specimen container 0007. The controlled temperatures of the heat source 2124 and the heat source 2126 are controlled such that the temperature of the housing unit 2001 is a temperature that does not affect the culture of the specimen 2111.

It is preferable that positions of the temperature sensors 2125 and 2127 are positions where a temperature of the specimen container lid 2112 or a space in the vicinity thereof can be relatively measured. In addition, as long as the correlation with the temperature of the specimen container lid 2112 in the housing section 0003 can be obtained, the temperature sensors 2125 and 2127 may be positioned at any position. The temperature sensors 2125 and 2127 may be positioned on a metal surface in the specimen container housing chamber 2003 or on cover surfaces of the heat source 2124 and 2126. That is, the positions of the temperature sensors 2125 and 2127 are not limited as long as the temperature of the specimen 2111 can be directly or indirectly measured.

In the above-described embodiments, the temperature control section 2002 may be disposed in the duct 2108. Note that, when a heat exchange mechanism such as a Peltier element is used as the temperature control section 2002, it is desirable that the position of the housing section 0003 and the like is appropriately adjusted such that the lower heat side (for example, the heat sink 2105) can be easily disposed outside the analysis device 0001.

### Third Embodiment

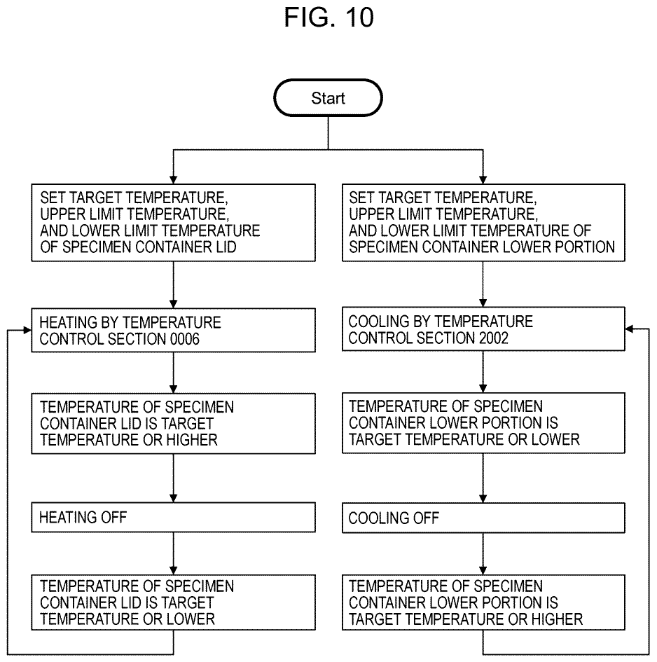

FIG. 10 is a flowchart illustrating a procedure where the arithmetic section 11 controls the temperature of each of sections in the analysis device 0001. The present flowchart can be used in the configuration of any of FIGS. 2 and 5 to 8. The present flowchart is executed by the arithmetic section 11 controlling each of the temperature control sections.

The temperature control (the left half part of the flowchart) of the entire analysis device 0001 by the temperature control section 0006 and the temperature control (the right half part of the flowchart) by the temperature control section 2002 can be simultaneously executed. The temperature control by the temperature control section 0006 is the temperature control of the entire device, in particular, mainly the temperature control for suppressing occurrence of dew condensation in the lid using the temperature control by the specimen container lid 2112 in the housing section 0003. The temperature control by the temperature control section 2002 is mainly the temperature control of the specimen using the temperature control of the specimen container lower portion 2110.

In the temperature control procedure of the entire device, a target temperature, an upper limit temperature, and a lower limit temperature of the specimen container lid 2112 in the housing section 0003 are set, and the specimen container lid 2112 is heated by the temperature control section 0006. The upper limit temperature or the lower limit temperature may be set based on the upper limit or the lower limit of the temperature that affects the growth or reaction of the specimen, or may be set depending on measurement conditions of the specimen measurement determined by a measurer. Note that the target temperature of the specimen container lid 2112 needs to be set to be higher than a target temperature of the specimen container lower portion 2110 such that the temperature of the specimen container lid 2112>the temperature of the specimen container lower portion 2110 is satisfied. The upper limit temperature and the lower limit temperature may be set as an allowable temperature range. The upper limit temperature and the lower limit temperature do not need to be set as an allowable temperature range.

In the heating procedure by the temperature control section 0006, when the temperature of the specimen container lid 2112 is the target temperature or higher, the heating is turned off. Note that, after turning off the heating, overshoot is set to be lower than the upper limit temperature, and undershoot is set to be the lower limit temperature or higher. Further, when the temperature of the specimen container lid 2112 is the target temperature or lower, the specimen container lid 2112 is heated by the temperature control section 0006. Instead of measuring the temperature of the specimen container lid 2112 itself, the temperature in the vicinity of the specimen container lid 2112 where the correlation can be obtained may be measured. That is, the configuration is not limited as long as the temperature of the specimen container lid 2112 can be directly or indirectly measured.

In the temperature control of the specimen container lower portion 2110, a target temperature, an upper limit temperature, and a lower limit temperature of the specimen container lower portion 2110 are set, and the specimen container lower portion 2110 is heated by the temperature control section 2002. The upper limit temperature or the lower limit temperature may be set based on the upper limit or the lower limit of the temperature that does not affect the specimen, or may be set depending on measurement conditions of the specimen measurement determined by a measurer. Note that the specimen temperature needs to be set to a temperature lower than the temperature of the specimen container lid 2112. In this flow, the temperature of the entire device is set through the temperature of the specimen container lid 2112. Therefore, the specimen container lower portion 2110 is cooled to be lower than the temperature of the specimen container lid 2112. Accordingly, the cooling by the temperature control section 2002 is shown in the flow.

In the cooling procedure by the temperature control section 2002, when the temperature of the specimen container lower portion 2110 is the target temperature or lower, the cooling is turned off. Note that, after turning off the cooling, undershoot is set to be the lower limit temperature or higher, and overshoot is set to be lower than the upper limit temperature. Further, when the temperature of the specimen container lower portion 2110 is the target temperature or higher, the specimen container lower portion 2110 is cooled by the temperature control section 2002. When the temperature of the specimen container lower portion 2110 is likely to be higher than the temperature of the specimen container lid 2112 depending on the set temperature, the cooling by the temperature control section 2002 needs to be controlled such that the temperature of the specimen container lid 2112>the temperature of the specimen container lower portion 2110 is satisfied.

A setting example of each of the temperatures is, for example, as follows: the target temperature, the upper limit temperature, and the lower limit temperature of the specimen container lid 2112 are 35.5 °C, 36.0 °C, and 35.0 °C, respectively; and the target temperature, the upper limit temperature, and the lower limit temperature of the specimen container lower portion 2110 are 34.5 °C, 35.0 °C, and 34.0 °C, respectively.

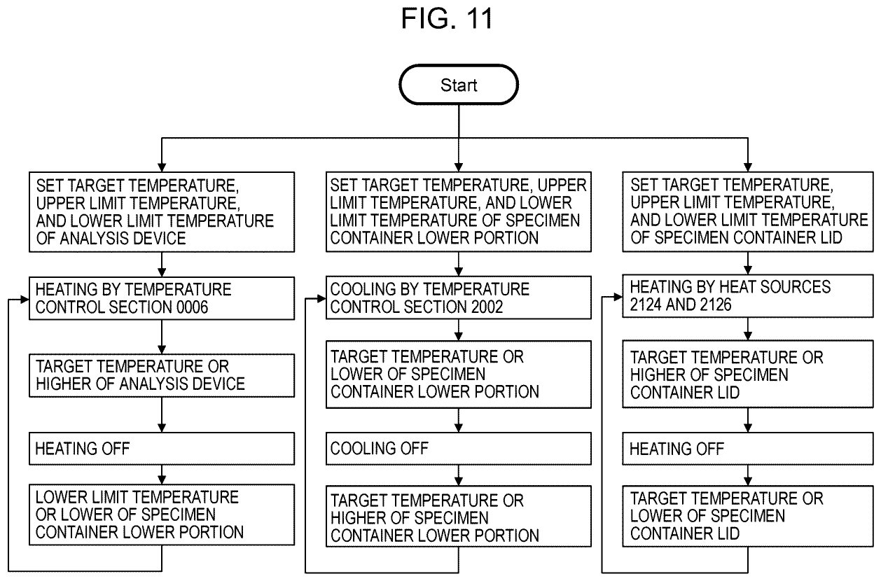

The lower limit temperature of the specimen container lid 2112 can be used, for example, for determining control parameters. For example, when one interval of one loop cycle of the left side of FIG. 11 is determined, the lower limit temperature of the specimen container lid 2112 may be used as a reference.

FIG. 11 is a flowchart illustrating a procedure where the arithmetic section 11 controls a temperature of each of sections in the analysis device 0001. The present flowchart can be used in the configuration of FIG. 9. The present flowchart is executed by the arithmetic section 11 controlling each of the temperature control sections.

The temperature control (the left of FIG. 11) of the entire analysis device 0001 by the temperature control section 0006, the temperature control (the center of FIG. 11) by the temperature control section 2002, and the temperature control (the right of FIG. 11) by the heat sources 2124 and 2126 can be simultaneously executed. The temperature control by the temperature control section 0006 can be mainly the temperature control of the entire analysis device 0001. The temperature control by the temperature control section 2002 is mainly the temperature control of the specimen container lower portion 2110 (specimen) in the housing section 0003. The temperature control by the heat sources 2124 and 2126 is mainly the temperature control for suppressing occurrence of dew condensation of the lid using the temperature control of the specimen container lid 2112. By executing the temperature control as described above, a higher temperature control accuracy can be implemented as compared to the temperature control of FIG. 10.

In the temperature control procedure of the entire device, a target temperature, an upper limit temperature, and a lower limit temperature of the analysis device 0001 are set, and the analysis device 0001 is heated by the temperature control section 0006. The upper limit temperature or the lower limit temperature may be set based on the upper limit or the lower limit of the temperature that affects the specimen, or may be set depending on measurement conditions of the specimen measurement determined by a measurer. The upper limit temperature and the lower limit temperature may be set as an allowable temperature range.

In the heating procedure by the temperature control section 0006, when the temperature of the analysis device 0001 is the target temperature or higher, the heating is turned off. When the temperature of the device is the lower limit temperature or lower of the specimen container lower portion 2110, the device is heated by the temperature control section 0006. In consideration of the influence of the internal temperature of the device when the specimen is transported or measured, a heating setting when the lower limit temperature or lower of the specimen container lower portion 2110 is reached is preferable. When a temperature effect during the transport or measurement of the specimen is not considered, the analysis device 0001 may be heated to be the target temperature or lower. Accordingly, depending on condition settings of the user, the step on the left side of the lowermost stage of FIG. 11 may be “the lower limit temperature or lower of the specimen container lower portion” or may be “the target temperature or lower of the analysis device”.

In the temperature control procedure by the temperature control section 2002, a target temperature (the target temperature of the specimen), an upper limit temperature, and a lower limit temperature of the specimen container lower portion 2110 are set, and the specimen container lower portion 2110 is heated by the temperature control section 2002. The upper limit temperature or the lower limit temperature may be set based on the upper limit or the lower limit of the temperature that does not affect the specimen, or may be set depending on measurement conditions of the specimen measurement determined by a measurer. Note that the temperature (specimen temperature) of the specimen container lower portion 2110 needs to be set to a temperature lower than the temperature of the specimen container lid 2112. In this flow, the specimen container lower portion 2110 is cooled to be lower than the temperature of the specimen container lid 2112. Therefore, the cooling by the temperature control section 2002 is shown in the flow.

In the temperature control by the temperature control section 2002, when the temperature of the specimen container lower portion 2110 is the target temperature or lower, the cooling is turned off. Note that, after turning off the cooling, undershoot is set to be the lower limit temperature or higher, and overshoot is set to be lower than the upper limit temperature. Further, when the temperature of the specimen container lower portion 2110 is the target temperature or higher, the specimen container lower portion 2110 is cooled by the temperature control section 2002.

In the temperature control of the specimen container lid 2112, a target temperature (the target temperature of the specimen container lower portion 2110), an upper limit temperature, and a lower limit temperature of the specimen container lid 2112 are set, and the specimen container lid 2112 is heated by the heat sources 2124 and 2126. The lower limit temperature of the specimen container lid 2112 is set such that the temperature of the specimen container lid 2112 can be controlled to be higher than the temperature of the specimen container lower portion 2110. That is, the lower limit temperature of the specimen container lid 2112 is not limited as long as the temperature of the specimen container lid 2112>the temperature of the specimen container lower portion 2110 can be reliably implemented.

In the temperature control procedure by the heat sources 2124 and 2126, when the temperature of the specimen container lid 2112 is the target temperature or higher, the heating is turned off. Note that, after turning off the heating, overshoot is set to be lower than the upper limit temperature, and undershoot is set to be the lower limit temperature or higher. Further, when the temperature of the specimen container lid 2112 is the target temperature or lower, the specimen container lid 2112 is heated by the heat sources 2124 and 2126. Since the heat source is positioned in the vicinity of the specimen container 0007, the temperature can be controlled with high accuracy. Accordingly, the temperature control can be executed in consideration of a temperature effect of the temperature control section 0006 or a temperature effect of the temperature control section 2002. When the temperature of the specimen container lower portion 2110 is higher than the temperature of the specimen container lid 2112 depending on the set temperature, the heating by the heat sources 2124 and 2126 needs to be controlled such that the temperature of the specimen container lid 2112>the temperature of the specimen container lower portion 2110 is satisfied.

A setting example of each of the temperatures is, for example, as follows: the target temperature, the upper limit temperature, and the lower limit temperature of the analysis device 0001 are 35.0 °C, 35.3 °C, and 34.5 °C, respectively; the target temperature, the upper limit temperature, and the lower limit temperature of the specimen container lower portion 2110 are 35.0 °C, 35.3 °C, and 34.5 °C, respectively; and the target temperature, the upper limit temperature, and the lower limit temperature of the specimen container lid 2112 are 35.5 °C, 36.0 °C, and 35.3 °C, respectively.

### Regarding Modification Example of Present Invention

The present invention is not limited to the embodiments and includes various modification examples. For example, the embodiments have been described in detail in order to easily describe the present invention, and the present invention is not necessarily to include all the configurations described above. In addition, a part of the configuration of one embodiment can be replaced with the configuration of another embodiment. Further, the configuration of one embodiment can be added to the configuration of another embodiment. In addition, addition, deletion, and replacement of another configuration can be made for a part of the configuration of each of the embodiments.

In the above-described embodiments, the outlet 2113 is configured by forming an opening in the entire surface of the specimen container housing chamber 2003, and the size and position of the opening of the air volume adjusting material (2116 or the like) are adjusted. As a result, the temperature of the specimen container lid 2112 can be set to be higher than the temperature of the specimen container lower portion 2110. That is, by combining the outlet 2113 and the air volume adjusting material (2116 or the like), the same effect as that of the above-described embodiments can be exhibited.

In the above-described embodiments, the arithmetic section 11 may present the temperature measured by each of the temperature sensors, the air volume (and/or air pressure) of each of the units, and the like on the monitor 13. Other information useful to the user may also be presented.

In the above-described embodiments, the arithmetic section 11 can also be configured by hardware such as a circuit device that implements the function or can also be configured by an arithmetic device such as a central processing unit (CPU) executing software that implements the function.

### REFERENCE SIGNS LIST

0001: analysis device
0002: loading/unloading section
0003: housing section
0004: transport section
0005: detection section
0006: temperature control section
0007: specimen container
1001: actuator
1002: actuator
1003: specimen container holding section
1004: measurement unit
1005: measurement section
1006: heat source
1007: heat sink
1008: fan
1009: temperature sensor
2001: housing unit
2002: temperature control section
2003: specimen container housing chamber
2101: cold source
2102: heat sink
2103: fan
2104: temperature sensor
2105: heat sink
2106: fan
2107: fan
2108: duct
2109: heat insulating material
2110: specimen container lower portion
2111: specimen
2112: specimen container lid
2113: outlet
2114: heat insulating material
2115: metal material
2116: air volume adjusting material
2117: metal material
2118: metal material
2119: air volume adjusting material
2120: duct
2121: air volume adjusting material
2122: air volume adjusting material
2123: heat insulating material
2124: heat source
2125: temperature sensor
2126: heat source
2127: temperature sensor

1. A specimen analysis device that analyzes a specimen, the specimen analysis device comprising:
a housing section configured to house a specimen container accommodating the specimen;
a temperature control section configured to generate cold air;
a first duct through which the cold air passes; and
an outlet from which the cold air passing through the first duct is supplied to a region below the specimen container in the housing section,
wherein the specimen container includes a specimen container lower portion and a specimen container lid that covers an upper surface of the specimen container lower portion, and
the temperature control section generates the cold air such that a temperature of the specimen container lower portion is lower than a temperature of the specimen container lid.

2. The specimen analysis device according to claim 1, further comprising a first air volume adjusting material configured to adjust an air volume of the cold air that is supplied to the housing section from the outlet.

3. The specimen analysis device according to claim 2,
wherein in the housing section, two or more housing chambers each of which houses the specimen container are disposed adjacent to each other,
the outlet is configured with two or more openings from which the cold air is supplied to each of the housing chambers, and
the first air volume adjusting material is configured to uniformize the air volume of the cold air supplied from each of the openings.

4. The specimen analysis device according to claim 1,
wherein the outlet is configured such that the cold air passes through a region below the specimen container lower portion.

5. The specimen analysis device according to claim 3,
wherein the outlet is configured to satisfy at least one of
a condition that the cold air is introduced into the housing chamber from a position below the specimen container lower portion,
or
a condition that an air volume of the cold air passing through a region below the specimen container lower portion is more than an air volume of the cold air passing through a region above the specimen container lower portion.

6. The specimen analysis device according to claim 3,
wherein the opening is configured to satisfy at least one of
a condition that the opening is provided only at a portion below the specimen container lower portion in an inlet surface where the cold air is introduced into the housing chamber,
or
a condition that a total area of the opening that is provided at the portion below the specimen container lower portion in the inlet surface where the cold air is introduced into the housing chamber is more than a total area of the opening that is provided at a portion above the specimen container lower portion.

7. The specimen analysis device according to claim 3,
wherein in the housing chamber, a heat insulating material is disposed below a portion where the specimen container is placed.

8. The specimen analysis device according to claim 1, further comprising:
a second duct configured to adjust a pressure of the cold air supplied to the housing section from the first duct; and
a second air volume adjusting material configured to adjust an air volume of the cold air, the second air volume adjusting material being disposed between the first duct and the second duct.

9. The specimen analysis device according to claim 2, further comprising:
a second duct configured to adjust a pressure of the cold air supplied to the housing section from the first duct; and
a second air volume adjusting material configured to adjust an air volume of the cold air, the second air volume adjusting material being disposed between the first duct and the second duct,
wherein an opening ratio of the first air volume adjusting material is different from an opening ratio of the second air volume adjusting material.

10. The specimen analysis device according to claim 8, further comprising a third air volume adjusting material configured to adjust an air volume of the cold air, the third air volume adjusting material being disposed between the second duct and the outlet,
wherein an opening ratio of the second air volume adjusting material is more than an opening ratio of the third air volume adjusting material.

11. The specimen analysis device according to claim 1,
wherein at least a part of an inner surface of the first duct or at least a part of an outer surface of the first duct is covered with a heat insulating material.

12. The specimen analysis device according to claim 1,
wherein the housing section includes a heat source and a temperature sensor on a side surface that is not orthogonal to a direction in which the cold air is introduced from the outlet, the temperature sensor being configured to measure a temperature of the heat source, and
a controlled temperature of the heat source is higher than a controlled temperature of the temperature control section.

13. The specimen analysis device according to claim 3, further comprising an air flow generating device configured to generate an air flow flowing through each of the housing chambers in the first duct, and
the temperature control section is disposed in the first duct.

14. The specimen analysis device according to claim 4,
wherein the temperature control section includes
a first temperature control device configured to control a temperature of the specimen container lid by supplying warm air into a housing of the specimen analysis device, and
a second temperature control device configured to control a temperature of the specimen container lower portion by supplying the cold air to the housing section,
the specimen analysis device further includes an arithmetic section configured to control the temperature control section, and
the arithmetic section controls the temperature control section such that the temperature of the specimen container lid is higher than the temperature of the specimen container lower portion.

15. The specimen analysis device according to claim 12,
wherein the temperature control section includes
a first temperature control device configured to control a temperature of the specimen container lower portion by supplying the cold air to the housing section, and
a second temperature control device configured to control a temperature of the specimen container lid by supplying warm air to the housing section using the heat source,
the specimen analysis device further includes an arithmetic section configured to control the temperature control section and the heat source, and
the arithmetic section controls the temperature control section and the heat source such that the temperature of the specimen container lid is higher than the temperature of the specimen container lower portion.

16. The specimen analysis device according to claim 1,
wherein the specimen is a biological body, and
the temperature control section supplies the cold air of a temperature at which the biological body is growable.
